(19) United States
(12) Patent Application Publication
(10) Pub. No.: US 2014/0271716 A1
(43) Pub. Date: Sep. 18, 2014

(54) Intra-testicular Injection of Immunogens

(71) Applicant: Min Wang, Columbia, MO (US)

(72) Inventor: Min Wang, Columbia, MO (US)

(73) Assignee: Ark Sciences, Inc., Baltimore, MD (US)

(21) Appl. No.: 13/837,755

(22) Filed: Mar. 15, 2013

(51) Int. Cl. A61K 9/00; A61K 31/315; A61K 39/205

(52) U.S. Cl. CPC A61K 9/0034; A61K 39/205; A61K 31/315; A61K 9/0019
USPC 424/224.1

(57) ABSTRACT

A method for inducing an immune response by injecting an immunogen into a subject's testis. A composition for intra-testicular injection including an immunogen such as a rabies vaccine and a chemical sterilant formed of zinc gluconate and an amino acid capable of forming an aqueous solution neutralized to a pH from 6.0 to 7.5. When injected intra-testicularly, the immunogen is slowly released reducing or eliminating the need for a “booster” dose, while the chemical sterilant is effective at reducing the dog population.

### BACKGROUND OF THE INVENTION

1. Field of the Invention

The present invention relates to intra-testicular injection of an immunogen capable of inducing an immune response. Said injection provides sustained stimulation of a subject's immune system through slow release of the immunogen into the subject's vascular system. Other medicinal products such as a chemical sterilant may be combined with the immunogen for intra-testicular injection.

2. Brief Description of the Prior Art

Vaccines, for example, induce an immune response when injected into a subject's body. It is known that subcutaneous injections of a vaccine can cause local reactions such as irritation, inflammation, granuloma formation and necrosis. For that reason, most vaccines are administered via an intramuscular route into the deltoid or the anterolateral aspect of the thigh. Muscle may be spared the harmful effects of substances injected into it because of its abundant blood supply which quickly disperses the vaccine into the subject's vascular system. The vaccine stimulates the subject's immune system to make germ fighting tools needed to fight an infection, some of which are kept in circulation after the immune response has been triggered. But in time, the immunity provided by the vaccine may wear off and a “booster” dose may be needed to bring the immunity levels back up. That requires a second intramuscular injection.

While effective rabies vaccines are available for intramuscular injection, rabies remains a serious problem in some countries. In Thailand, for example, stray and community dogs are the main vectors for rabies and left untreated, most rabies dog-bite victims die, and many of whom are children. There are expensive post-exposure treatments, but in many areas post-exposure treatment is not available. To control rabies, it has been found that from 60 to 80% of the dogs must be rabies vaccinated. To reach that goal in a population of stray and community dogs within an affordable budget, it may be necessary to reduce the number of dogs. But cultural barriers may prevent large scale culling of dogs to facilitate vaccination of enough dogs in the dog population for rabies elimination. When not enough dogs are vaccinated to eliminate rabies from the dog population, it is necessary to administer a “booster” dose to the immunized dogs an interval of three years or less which greatly adds to the cost of controlling rabies.

### BRIEF SUMMARY OF THE INVENTION

In accordance with the present invention, it is disclosed that the pharmakinetic release of an immunogen that is intra-testicularly injected extends over a longer period of time than when injected intramuscularly thus providing for sustained stimulation of a subject's immune system. One composition for intra-testicular injection comprises a chemical sterilant and an immunogen capable of inducing an immune response wherein the chemical sterilant is zinc gluconate and an amino acid capable of forming an aqueous solution, said zinc gluconate and amino acid being present in substantially equal molar amounts and at a concentration in the range from about 0.05 M to 2.0 M and neutralized to a pH from about 6.0 to about 7.5. When the immunogen is a rabies vaccine and combined with a chemical sterilant, less rabies vaccine may need to be injected to effect inoculation against rabies. Included among the methods disclosed is one for forming the above-mentioned composition when the immunogen is a dried inactivated rabies vaccine. In which case, the composition must be injected immediately after being formed or stored under refrigeration.

The invention summarized above comprises the compositions and methods hereinafter described, the scope of the invention being indicated by the subjoined claims.

### BRIEF DESCRIPTION OF THE SEVERAL VIEWS OF THE DRAWINGS

FIG. 1 is graph showing the zinc level in blood periodically collected after intramuscular injection with zinc acetate;

FIG. 2 is a graph showing the zinc level in blood periodically collected after intra-testicular injection with zinc acetate; and,

FIG. 3 is a chart showing the rabies antibody titer over a period of four weeks after intra-testicular injection with Esteril™ and Placebo, Rabies Vaccine and Placebo and Esterilsol and Rabies Vaccine.

### DETAILED DESCRIPTION OF THE INVENTION

An immunogen is a substance capable of inducing an immune response when injected into a host's body. Vaccine immunogens, for example, typically contain an agent that resembles a disease-causing microorganism, and is often made from weakened or killed forms of the microbe, its toxins or one of its surface proteins. The agent stimulates the body's immune system to recognize the agent as foreign, destroy it and remember it, so that the immune system can more easily recognize and destroy any of the microorganisms that it later encounters. Injection with a vaccine does not guarantee complete protection from the disease but, in general, when a vaccinated individual does develop the disease vaccinated against, the disease is likely to be milder than without vaccination. Included among the diseases which may be treated with a vaccine immunogen are Anthrax, Diphtheria, Haemophilus Influenzae type b (Hib), Hepatitis A, Hepatitis B, Human Papillomavirus (HPV), Influenza, Japanese Encephalitis, Lyme Disease, Measles, Meningococcal, Mumps, Pertussis (Whooping Cough), Pneumococcal Disease, Polio, Rabies, Rotavirus, Rubella, Shingles (Herpes Zoster), Smallpox, Tetanus, Tuberculosis, Typhoid Fever, Varicella (Chickenpox) and Yellow Fever.

Combination vaccines merge immunogens that prevent different diseases into a single product or that protect against multiple strains of infectious agents causing the same disease. Thus, they reduce the number of injections required to prevent some diseases. Representative combination vaccines include diphtheria and tetanus toxoids and whole-cell pertussis vaccine (DDTwP); measles-mumps-rubella vaccine (MMR); and trivalent inactivated polio vaccine (IPV). Other combinations licensed in the United States include diphtheria and tetanus toxoids and acellular pertussis vaccine (DTaP), DTwP-Haemophilus influenzae type b (Hib) vaccine (DTwP-Hib), DTaP-Hib, and Hib-hepatitis B (HepB) vaccine (Hib-HepB). Combination vaccines like single strain vaccines may be candidates for intra-testicular injection.

Applicant's work has focused on the intra-testicular injection of a rabies vaccine combined with a chemical sterilant into dogs but the invention has application to other species with scrotal testes including humans and to the injection of an immunogen without a chemical sterilant.

Rabies vaccines for prophylactic vaccination of dogs that are suitable for intra-testicular injection may contain an inactivated rabies virus or a live attenuated virus. Most of the rabies vaccines used today contain an inactivated rabies virus. Several manufacturers provide combination vaccines which include a variety of different antigens (e.g., distemper, adenovirus, leptospirosis, parainfluenza, parvovius, etc.) along with the rabies immunogen. Live attenuated virus vaccines, such as LEP (low egg passage), HEP (high egg passage) and ERA (Evelyn Rokitniki Abelseth) have been used in the past and recombinant vaccines and other products of genetic engineering may also be used.

Many commercially available rabies vaccines are supplied in a dried form and after being reconstituted require refrigeration or should be discarded. For example, the Imovax™ Rabies Vaccine produced by Sanofi Pasteur SA is a sterile, stable, freeze-dried suspension of rabies virus and is provided for intramuscular in a single dose vial containing no preservative. After reconstitution, the company's instructions provide that the full 1.0 ml amount of vaccine should be immediately injected intramuscularly and if not administered promptly, discarded. For intra-testicular injection, the amount of rabies vaccine injected into the testes may be comparable to the amount recommended for intramuscular injection, although as shown in Example 3a lesser amount may be necessary.

A chemical sterilant for use in the present invention in combination with an immunogen is disclosed in U.S. Pat. No. 5,070,080 to Fahim and a preferred method of injecting of the chemical sterilant is disclosed in U.S. Pat. No. 7,276,535 to Wang. The chemical sterilant described in the '080 patent is a zinc gluconate salt and an amino acid capable of forming an aqueous solution neutralized with an acid to a pH in the range of 6.0 to 8.0, preferably from about 6.0 to about 7.5 and most preferably about 7.0. Suitable amino acids for neutralizing the zinc gluconate include alanine, valine, isoleucine, proline, glycine, serine, threonine, asparagine, glutamine, lysine, arginine, histidine and mixtures thereof.

In neutralizing zinc gluconate, it is preferred that the zinc gluconate and the amino acid be present in substantially eqimolar amounts and it is desirable that the smallest possible effective amount of the chemical sterilant be injected into the testis. In the '080 patent, the chemical sterilant was injected into the midline of the testis from the side or bottom. But as disclosed in the '535 patent, the dose may be minimized by injecting the chemical sterilant into the dorsal cranial portion of the testes beside the epididymis. The use of the chemical sterilant for controlling dog population is described in International Publication No. WO 2009/045337 A1 to Wang. As disclosed, the chemical sterilant effects sterilization without effecting the sterilized dog's position in a community of dogs. The sterilized dog “breeds” with receptive females but no puppies result and in time the dog population declines.

The combination of a chemical sterilant and a rabies vaccine and the method described above reduces the population of dogs in a community by reducing the breeding effectiveness of the treated males. It also provides for sustained release of the rabies vaccine for continued stimulation of the dog's immune response to rabies thereby reducing (or eliminating) the need for a “booster” dose. This combination of effects may place the control of rabies within the budget of even a developing country.

The following data illustrate the invention wherein an immunogen is injected intra-testicularly.

### Example 1

Six mixed Duroc pigs, three male and three female, 40 days old, and having an average weight of 15 kg were intramuscularly injected with 30 mg/kg of zinc acetate in the left shoulder. Blood was periodically collected from the jugular vein until the zinc level in the blood reached base line as shown in Table 1, data from which is plotted in FIG. 1.

TABLE 1

| | Concentration (μ mol/L) | | | | | | | | | | | | |
|---|---|---|---|---|---|---|---|---|---|---|---|---|---|
| | Time | | | | | | | | | | | | |
| | 0 Min | 30 min | 1 hr. | 2 hr. | 3 hr. | 4 hr. | 6 hr. | 8 hr. | 12 hr. | 16 hr. | 24 hr. | 36 hr. | 48 hr. |
| Zinc | 45.3 | 194.08 | 76.17 | 67.4 | 65.22 | 62.8 | 61.6 | 60.8 | 54.3 | 48.7 | 47.3 | 44.5 | 44.2 |
| Concentration | | | | | | | | | | | | | |

Six male Yorkshire pigs, 25 days old, and having an average weight of 12 kg were intra-testicularly injected with 0.5 ml (74 mg/ml of zinc acetate) into each testis. Blood was periodically collected from the jugular vein until the zinc level in the blood reached base line as shown in Table 2, data from which is plotted in FIG. 2.

TABLE 2

| | Concentration (μ mol/L) | | | | | | | | | | | | |
|---|---|---|---|---|---|---|---|---|---|---|---|---|---|
| | Time | | | | | | | | | | | | |
| | 0 Min | 30 Min | 1 hr. | 2 hr. | 3 hr. | 4 hr. | 6 hr. | 8 hr. | 12 hr. | 24 hr. | 48 hr. | 72 hr. | 96 hr. |
| Zinc | 0 | 8.44 | 7.49 | 5.65 | 5.27 | 4.96 | 4.06 | 4.43 | 3.87 | 4.43 | 3.11 | −0.36 | −0.29 |
| Concentration | | | | | | | | | | | | | |

Whether injected intramuscularly or intra-testicularly, the zinc was rapidly absorbed after injection and peaked between 30 and 60 minutes. In the pigs injected intramuscularly, the zinc concentration in the plasma returned to physiological baseline between 36 and 48 hours. With the pigs injected intra-testicularly, there was a second absorption phase 24 hours after injection producing a mean residence time of nearly 60 hours. The zinc concentration returned to baseline between 48 and 72 hours after injection

The two studies were conducted in two different laboratories and adopted different ways of expressing the amount of zinc in the blood at zero time. In the intra-testicular study, the zinc in the blood was taken at zero at time zero. In the intramuscular study, zinc in the blood was the physiological level at time zero.

### Example 2

Eighteen male dogs of mixed breeds were acquired from dog round-ups conducted by the Navajo Nation Animal Control Program during July 2010. Unclaimed dogs gathered by Animal Control are euthanized 3 days post round-up pursuant to the Navajo National Animal Control Laws (Navajo Tribal Code; Title 13, Section 1711, Impounded Animals). Male dogs over 3 months of age were selected for this study instead of euthanasia. Each dog was individually marked with an identification tag and all of the dogs were sedated and blood was collected as base day. All of the injections were completed according to the procedure on the product package insert and distilled water was used as a placebo in Groups A and B. The dogs were housed in standard commercial canine runs of sufficient size to allow free movement. All of the dogs were retained for observation at the investigation facility. Water was made available ad libitum and standard commercial dry dog food was also available. No other medicine or procedure was used in the study. A staff veterinary monitored the dogs for the entire investigation period. The blood samples were collected on a weekly basis and at the end of the study, all of the dog's sex organs were examined.

The dogs were divided into the following groups:

Group A: Six animals. All were injected intratesticularly with Esterilsol™ and an injection of placebo administered intramuscularly to the upper right hind leg.
Group B: Six animals. All were vaccinated with a single 1 ml injection of DEFENSOR-3 rabies vaccination, administered intramuscularly to the upper right hind leg and an intratesticular injection of placebo.
Group C: Six animals. All were injected intratesticularly with Esterilsol and a single 1 ml injection of DEFENSOR-3 rabies vaccination administered intramuscularly to the upper right hind leg.

Esterilsol™ (Ark Sciences, Inc., Baltimore, Md., USA) consisted of zinc gluconate neutralized by 1-arginine. Each 2-ml vial contained 13.1 mg/ml of zinc gluconate and 34.1 mg/ml of arginine stored at room temperature.

The rabies virus vaccine was a commercially available inactivated rabies virus (DEFENSOR 3, Pfizer, Inc., New York, N.Y., USA). Each 1 ml container was stored under refrigerated conditions at 4 until ready for use.

### Determination of Esterilsol™ Efficacy

At Day 33, the testes and epididymides were remove from all animals and fixed in neutral buffered 10% formalin, embedded in paraffin, sectioned at 4 μm, stained with hematoxylin and eosin for histopathological evaluation. The organs were sent to the University of Missouri College of Veterinary Medicine for complete evaluation.

### Determination of Rabies VNA Titers

Blood was drawn from the jugular vein of each dog on a weekly basis using a 12 ml syringe equipped with a 20-gauge needle. Blood samples were stored on blue ice in an ice chest and then centrifuged. Blood samples were sent to the Centers for Disease Control in Atlanta, Ga. for analysis. The coded sera were thawed rapidly and heat-inactivated in 56° C. water bath for 1 h. Rabies VNA titers were determined using the Rapid Fluorescent Focus Inhibition Tests.

### Results

All of dogs were healthy; no major general complications were noted during the post-injection follow-up periods. Testicular and epididymal histopathology report showed that Group B which received the rabies vaccine only had all of the stages of the seminiferous epithelium, as well as all of phases of spermatid development are identified. Sperm were present within the epididymis. Groups A and C which received Esterilsol™ had severe bilateral degeneration of most of the seminiferous tubules, with lymphocytic infiltration and disruption of portions of the interstitum. The segments of rete testes and efferent ductules examined appear to have under gone some degeneration. No sperm were observed in any of sections of the epididymides.

The rabies VNA titers for each group were determined over 33 day period. The titers are show in FIG. 3. All of dogs in Groups B and group C, which received the rabies vaccination, had response to the rabies vaccine indicating no cross-interference of the effectiveness of rabies vaccination with Esterilsol™.

### Example 3

Forty SD sexually mature male rats were divided into four groups of ten rats per group:

Group 1: Injected with 0.05 ml rabies vaccine⁽¹⁾ into each testis.

Group 2: Injected with 0.1 ml ZEUTERIN™ plus 0.1 ml of rabies vaccine into each testis.

Group 3: Injected with 0.1 ml rabies vaccine intramuscularly.

Group 4: Injection with 0.1 ml ZEUTERIN™ plus 0.05 rabies vaccine into each testis.

ZEUTERIN™ (Ark Sciences, Inc., Baltimore, Md., USA) is an aqueous solution containing 13.1 mg/ml of zinc as zinc gluconate neutralized by 34.8 mg/ml of 1-arginine with the pH adjusted to 7.0 with hydrochloric acid. The rabies virus vaccine was a commercially available inactivated rabies virus (DEFENSOR 3, Pfizer, Inc., New York, N.Y., USA) stored under refrigeration until ready for use.

The results are given in the following tables.

TABLE 3
Body Weights (g) (End of day)

| No. | G1 | G2 | G3 | G4 |
|---|---|---|---|---|
| 1 | 423 | 464 | 390 | 345 |
| 2 | 375 | 433 | 390 | 405 |
| 3 | 440 | 346 | 350 | 350 |
| 4 | 468 | 383 | 380 | 410 |
| 5 | 450 | 340 | 400 | 397 |
| 6 | 420 | 320 | 420 | 345 |
| 7 | 400 | 420 | 390 | 358 |
| 8 | 460 | 375 | 360 | 400 |
| 9 | 420 | 396 | 362 | 395 |
| 10 | 375 | 350 | 466 | 310 |
| X | 423.1* | 382.7 | 390.8 | 371.5 |
| SD | 32.60 | 45.87 | 33.58 | 34.07 |

*P < 0.05 comparison with group 3

TABLE 4
Weights of Testis (g)

| No. | G1 | G2 | G3 | G4 |
|---|---|---|---|---|
| 1 | 4.2 | 1.297 | 4.27 | 1.361 |
| 2 | 4.36 | 1.485 | 3.288 | 0.759 |
| 3 | 4.55 | 0.495 | 4.619 | 0.935 |
| 4 | 4.34 | 1.342 | 3.63 | 0.971 |
| 5 | 4.21 | 1.129 | 2.954 | 0.812 |
| 6 | 4.34 | 1.454 | 4.6 | 1.079 |
| 7 | 4.18 | 2.522 | 4.29 | 1.071 |
| 8 | 4.25 | 1.446 | 5.31 | 1.412 |
| 9 | 4.68 | 1.714 | 5.1 | 1.303 |
| 10 | 4.3 | 2.11 | 4.33 | 1.252 |
| X | 4.341 | 1.4994* | 4.2391 | 1.0955* |
| SO | 0.1605 | 0.5458 | 0.7528 | 0.2294 |

*P < 0.0001 comparison with group 3

TABLE 5
Concentration of RV-Ab (pg/ml) serum

| No. | G1 | G2 | G3 | G4 |
|---|---|---|---|---|
| 1 | 32.5055 | 37.357 | 40.9895 | 37.348 |
| 2 | 33.0405 | 26.3155 | 23.6255 | 30.3505 |
| 3 | 29.0055 | 31.695 | 28.109 | 25.867 |
| 4 | 36.178 | 35.282 | 34.3855 | 33.937 |
| 5 | 33.0405 | 30.3505 | 36.178 | 21.832 |
| 6 | 28.1085 | 28.1085 | 26.7635 | 34.3855 |
| 7 | 33.937 | 28.557 | 35.7305 | 32.1435 |
| 8 | 27.212 | 29.0055 | 34.3855 | 38.4205 |
| 9 | 25.419 | 20.038 | 24.522 | 32.1435 |
| 10 | 38.869 | 26.3155 | 34.833 | 29.902 |
| X | 31.7315 | 29.3024 | 31.9522 | 31.6329 |
| SD | 4.2221 | 4.8670 | 5.7752 | 5.0121 |

TABLE 6
Concentration of RV-Ab (pg/ml) plasm

| No. | G1 | G2 | G3 | G4 |
|---|---|---|---|---|
| 1 | 28.8595 | 31.898 | 33.1115 | 39.3755 |
| 2 | 27.954 | 34.7995 | 35.361 | 33.0855 |
| 3 | 31.375 | 32.519 | 29.659 | 40.992 |
| 4 | 38.1605 | 29.083 | 25.653 | 44.355 |
| 5 | 29.099 | 34.794 | 34.221 | 36.493 |
| 6 | 37.622 | 31.954 | 35.361 | 40.9855 |
| 7 | 42.113 | 34.2275 | 32.506 | 39.296 |
| 8 | 38.182 | 34.2255 | 32.5225 | 47.139 |
| 9 | 25.653 | 38.186 | 32.5235 | 44.313 |
| 10 | 27.3745 | 38.1855 | 39.3085 | 42.678 |
| X | 32.6392 | 33.9872 | 33.0227 | 40.8712 |
| SD | 5.7992 | 0.5632 | 3.6252 | 4.0969 |

*P < 0.001 comparison with group 3

TABLE 7
Comparision of RV-Ab

| | Group | | | |
|---|---|---|---|---|
| | Group 1 | Group 2 | Group 3 | Group 4 |
| concentration of | 32.6392 ± 5.7992 | 33.9872 ± 0.5632 | 33.0227 ± 3.6252 | 40.87125 ± 4.0969 |
| RV-AB (plasm) (pg/ml) | | | | |
| P value | 0.8613 | 0.4167 | | 0.0003 |
| concentration of | 31.7315 ± 4.2221 | 29.30245 ± 4.8670 | 31.9522 ± 5.7752 | 31.6329 ± 5.0120 |
| RV-Ab (serum) (pg/ml) | | | | |
| P value | 0.9234 | 0.2818 | | 0.8964 |

P value: all the groups compare with group 3

As various changes could be made in the above compositions and methods without departing from the scope of the invention, it is intended that all matter contained in the above description and accompanying examples shall be interpreted as illustrative and not in a limiting sense.

What is claimed:

1. A method for inducing an immune response comprising
forming a solution of an immunogen capable of inducing an immune response when injected intramuscularly into a subject's body, said solution having a pH between about 6.0 and 7.5; and,
injecting said solution into each testis of a subject.

2. The method of claim 1 wherein the injection is into the dorsal cranial portion of the testis beside the epididymis.

3. The method of claim 1 wherein the amount of solution injected is that amount of immunogen effective to induce an immune response when injected intramuscularly.

4. The method of claim 3 wherein the immunogen is a vaccine.

5. A composition for intra-testicular injection comprising a chemical sterilant and an immunogen capable of inducing an immune response, said chemical sterilant comprising zinc gluconate and an amino acid capable of forming an aqueous solution, said zinc gluconate and amino acid being present in substantially equal molar amounts and at a concentration in the range from about 0.05 M to 2.0 M and neutralized to a pH from about 6.0 to about 7.5.

6. The composition of claim 5 for dog population and rabies control wherein the immunogen is a rabies vaccine.

7. The composition of claim 6 wherein the rabies vaccine contains an inactivated rabies virus.

8. The composition of claim 7 wherein the chemical sterilant aqueous solution is adjusted to about pH 7.0 and contains 13.1 mg/ml zinc gluconate and 34.8 mg/ml l-arginine.

9. A method for forming a composition for intra-testicular injection for dog population and rabies control comprising
preparing a chemical sterilant comprising zinc gluconate and an amino acid capable of forming an aqueous solution, said zinc gluconate and amino acid being present in substantially equal molar amounts and at a concentration in the range from about 0.05 M to 2.0 M and neutralized to a pH between about 6.0 and about 7.5;
reconstituting a dried inactivated rabies vaccine;
combining the chemical sterilant and reconstituted inactivated rabies vaccine for intra-testicular injection immediately after the inactivated rabies vaccine has been reconstituted or refrigerating the combination of chemical sterilant and reconstituted inactivated rabies vaccine for later injection.

10. A method for administering a chemical sterilant and a rabies vaccine to male dogs for population and rabies control, said method comprising,
preparing a chemical sterilant comprising zinc gluconate and an amino acid capable of forming an aqueous solution, said zinc gluconate and amino acid being present in substantially equal molar amounts and at a concentration in the range from about 0.05 M to 1.0 M and neutralized to a pH between about 6.0 and about 7.5;
reconstituting a dried inactivated rabies vaccine;
combining the chemical sterilant and reconstituted inactivated rabies vaccine;
injecting the combination of chemical sterilant and inactivated rabies vaccine into the dog's testes, said chemical sterilant present in an amount sufficient to render the dog sterile and the inactivated rabies vaccine present in an amount sufficient to inoculate the dog against rabies.

11. The method of claim 10 wherein the chemical sterilant aqueous solution is adjusted to about pH 7.0 and contains 13.1 mg/ml zinc gluconate and 34.8 mg/ml l-arginine.

12. The method of claim 11 wherein the intra-testicular injection is into a dorsal cranial portion of the testes beside the epididymis.
